(19) United States
(12) Patent Application Publication
(10) Pub. No.: US 2021/0362939 A1
(43) Pub. Date: Nov. 25, 2021

(54) AN EARPLUG DISPENSING SYSTEM

(71) Applicant: SwedSafe AB, HÖÖR (SE)

(72) Inventor: Jörgen Håkansson, TYRINGE (SE)

(21) Appl. No.: 16/955,925

(22) PCT Filed: Dec. 4, 2018
(86) PCT No.: PCT/EP2018/083476

§ 371 Date: Jun. 19, 2020

(30) Foreign Application Priority Data
Dec. 22, 2017 (SE) 1751627-9

(51) Int. Cl. B65D 83/04; F16B 1/00

(52) U.S. Cl. CPC B65D 83/0409; B65D 2583/0436; F16B 2001/0035; F16B 1/00

(57) ABSTRACT

An earplug dispensing system (10), comprising an earplug dispenser (11) and a support plate (12), wherein the dispenser (11) comprises a container (13) for earplugs, a dispensing mechanism having a dispensing aperture (14) and a movable element operably arranged for selectively dispensing earplugs from the container (13) through the aperture (14), and a control device (15) for operating the dispensing mechanism, wherein the support plate (12) on a front side (17) is provided with the earplug dispenser (11) and on a back side (19) is provided with one or more fastening devices (20) for fastening the support plate (12) to a vertical surface. The fastening devices (20) comprise a permanent magnet (21) for fastening the system to the vertical surface, wherein the magnet (21) has a back side provided with a friction-increasing material (22) for preventing the earplug dispensing system (10) from displacement in a plane of the vertical surface.

### FIELD OF THE INVENTION

The invention relates to an earplug dispensing system. In particular the present invention relates to an earplug dispensing system, comprising an earplug dispenser and a support plate having a front side provided with the earplug dispenser and on back side provided with one or more fastening devices for fastening the support plate to a vertical surface. Earplugs are generally used to protect auditory organs of a user being in environments with harmful sound. One common type of earplug is a foam plug or similar. This type of earplug dispensing systems is generally used for providing earplugs to staff in environments with increased noise levels, such as in environments with occupational noise or industrial noise. For example, such earplug dispensing systems are used in buildings with machines causing industrial noise or in building areas where staff pass before entering a noisy area or noisy vehicle.

### PRIOR ART

There are several different types of earplug dispensers in the prior art. One such type of earplug dispenser comprises an earplug dispenser with a container for a plurality of earplugs, a dispensing mechanism having a dispensing aperture with a closing element operably arranged between a closed position for closing the aperture and an open position for dispensing earplugs from the container through the aperture, and a control device for operating the dispensing mechanism. The earplug dispenser is attachable to a mounting bracket for carrying the earplug dispenser, wherein the mounting bracket can be fastened to a vertical surface in the form of a wall or similar.

There is always a need to further improve such earplug dispensing systems.

A problem with such earplug dispensing systems according to prior art is that they can be difficult to mount.

### SUMMARY OF THE INVENTION

An object of the present invention is to provide an improved earplug dispensing system. The present invention relates to an earplug dispensing system, comprising an earplug dispenser and a support plate for carrying the earplug dispenser, wherein the earplug dispenser comprises a container for a plurality of earplugs, a dispensing mechanism having a dispensing aperture with a closing element operably arranged between a closed position for closing the aperture and an open position for dispensing earplugs from the container through the aperture, and a control device for operating the dispensing mechanism, wherein the support plate on a front side is provided with the earplug dispenser and on a back side is provided with one or more fastening devices for fastening the support plate to a vertical surface, characterised in that the one or more fastening devices comprise a permanent magnet for fastening the support plate with the earplug dispenser to the vertical surface, wherein the magnet has a back side arranged substantially in parallel to the back side of the support plate, and that at least the back side of the permanent magnet is provided with a friction-increasing material for engaging the vertical surface and preventing the earplug dispensing system from displacement in a plane of the vertical surface.

The earplug dispensing system according to the invention results in facilitated mounting and moving of the earplug dispensing system. The combination of the permanent magnets and the increased-friction material results in easy and reliable mounting on magnetic vertical surfaces, including vertical surfaces on a wide range of common objects in industry, such as steel doors and walls, vertical machine surfaces, lockers, whiteboards, etc., as well as vertical magnetic vehicle surfaces, wherein the magnet fastener keeps the earplug dispensing system to the vertical surface and the increased-friction material prevents the earplug dispensing system from sliding along the vertical surface.

The back side of the support plate can be provided with a plurality of such permanent magnets, which can be distributed over the surface of the back side of the support plate, such as at each corner of a substantially rectangular or square support plate, to provide desired fastening of the earplug dispensing system to the vertical surface.

The permanent magnet can be arranged as a plate having a front side engaging the back side of the support plate and being arranged in parallel to the back side of the permanent magnet. A plate-shaped magnet can provide a considerable surface area of the permanent magnet to achieve both considerable magnetic forces over a large surface area as well as considerable frictional forces. For example, the total surface area of at least the back side of the magnets can be at least 20 cm² or at least 50 cm² to provide a favourable combination of magnetic and frictional forces. The entire surface of the back side of the magnets can be provided with the friction-increasing material. The friction-increasing material can be arranged as a sheet of friction-increasing material on the back side of the magnets, such as a sheet of elastomer material, e.g. in the form of rubber or other soft and high-friction material for preventing sliding of the magnets along the vertical surface.

The dispenser can be attached to the support plate through a mounting bracket, wherein the dispenser can be detachably attachable to the mounting bracket. Hence, both the whole earplug dispensing system and, if desired, only the dispenser can easily be removed.

The dispenser can be arranged substantially vertically, so that the earplugs are dispensed downwards by means of gravity. Hence, the earplugs can be stored in the container in an upright position to save space in the horizontal direction and minimize the ear dispensing system as a potential obstacle in industrial areas, vehicles, control rooms, locker rooms, corridors, etc. Hence, a considerable amount of earplugs can be stored in an elongated and vertically extending container without taking up useful space, while at the same time a reliable dispensing mechanism can be achieved as the earplugs are fed in a confined small area to the dispensing mechanism by means of gravity.

Further characteristics and advantages of the present invention will become apparent from the description of the embodiments below, the appended drawings and the dependent claims.

### SHORT DESCRIPTION OF THE DRAWINGS

The invention will now be described more in detail with the aid of embodiments and with reference to the appended drawings, in which

FIG. 1 is a schematic front view of an earplug dispensing system according to one embodiment, illustrating an earplug container, an earplug dispenser and a support plate of the earplug dispensing system,

FIG. 2 is a schematic perspective view obliquely from a back of the earplug dispensing system according to FIG. 1, illustrating a back side of the support plate with fastening devices according to one embodiment,

FIG. 3 is a schematic rear view of the earplug dispensing system according to FIG. 1, illustrating the back side of the support plate with the fastening devices,

FIG. 4 is a schematic front view of a part of the earplug dispensing system, wherein the earplug dispenser has been removed to illustrate a mounting bracket attached to the support plate,

FIG. 5 is a schematic side view of the support plate and the mounting bracket according to FIG. 4, wherein the support plate is attached to a surface of a vertical element, and

FIG. 6 is a schematic perspective view obliquely from above of the earplug dispenser, illustrating a dispensing mechanism thereof according to one embodiment.

### THE INVENTION

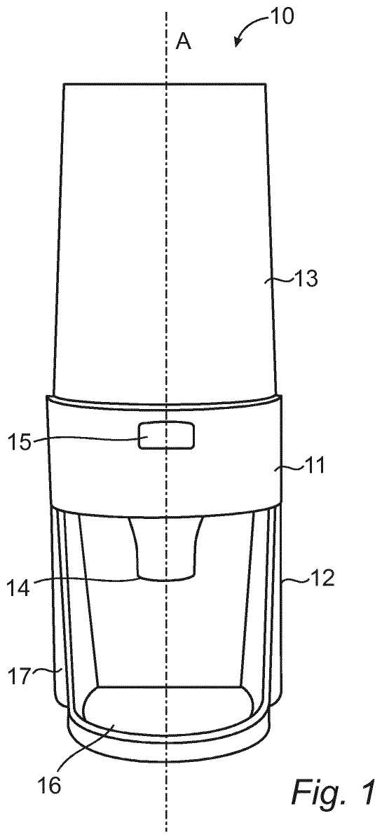

With reference to FIGS. 1-3 an earplug dispensing system 10 is illustrated according to one embodiment. The earplug dispensing system 10 is arranged to be mounted on a surface of a substantially vertical support element comprising a ferromagnetic material, also called a magnetic material herein. For example, the earplug dispensing system 10 is arranged to be mounted on a vertical element comprising iron, nickel, cobalt or other metals, alloys and/or minerals to which a magnet is attachable by magnetism to the surface thereof. The earplug dispensing system 10 is, for example arranged to be mounted to the surface of a vertical element in the form of a metal wall, plate, door, board or similar. For example, the earplug dispensing system is arranged to be mounted on a locker, whiteboard, machine part, vehicle part or similar.

The earplug dispensing system 10 comprises a longitudinal axis A. The earplug dispensing system 10 is configured to be arranged in an upright position, wherein the longitudinal axis A extends in a substantially vertical direction, so that earplugs are dispensed downwards in the vertical direction. In the illustrated embodiment, the earplug dispensing system 10 is elongated and extends along the longitudinal axis A.

The earplug dispensing system 10 comprises an earplug dispenser 11 and a support plate 12 for carrying the earplug dispenser 11. The earplug dispenser 11 comprises a container 13 for a plurality of earplugs, wherein a plurality of earplugs, such as hundreds of earplugs, can be stored in the container 13. The container 13 is arranged in a standing position for feeding earplugs downwards by means of gravity. For example, the container 13 is arranged substantially vertically when the earplug dispensing system 10 is mounted on a a vertical surface. For example, the container 13 is elongated and extends in the longitudinal direction A. According to one embodiment, the container 13 is made of plastic materials and is, e.g. transparent so that the level of earplugs therein is visible.

The earplug dispenser 11 further comprises a dispensing mechanism having a dispensing aperture 14. The dispensing mechanism is not illustrated in detail in the drawings. The dispensing mechanism is arranged with a movable element operably arranged for selectively dispensing earplugs from the container 13 through the aperture 14. The dispensing mechanism is operable by means of a control device 15 for operating the dispensing mechanism. For example, the control device 15 is a push button, a lever or similar. The dispensing mechanism and the aperture 14 are arranged in the lower part of the container 13 or below the container 13. According to one embodiment, the dispensing mechanism is conventional.

In the illustrated embodiment, the earplug dispensing system 10 comprises a tray 16 for receiving earplugs after exiting the earplug dispenser 11. The tray 16 is arranged below the container 13, the dispensing mechanism and the dispensing aperture 14. For example, the tray 16 is arranged in a plane extending substantially in parallel to a plane of the dispensing aperture 14 and perpendicular to the longitudinal axis A.

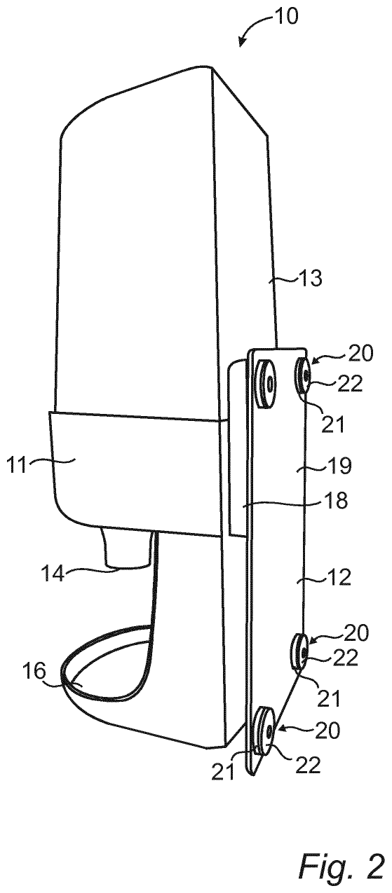

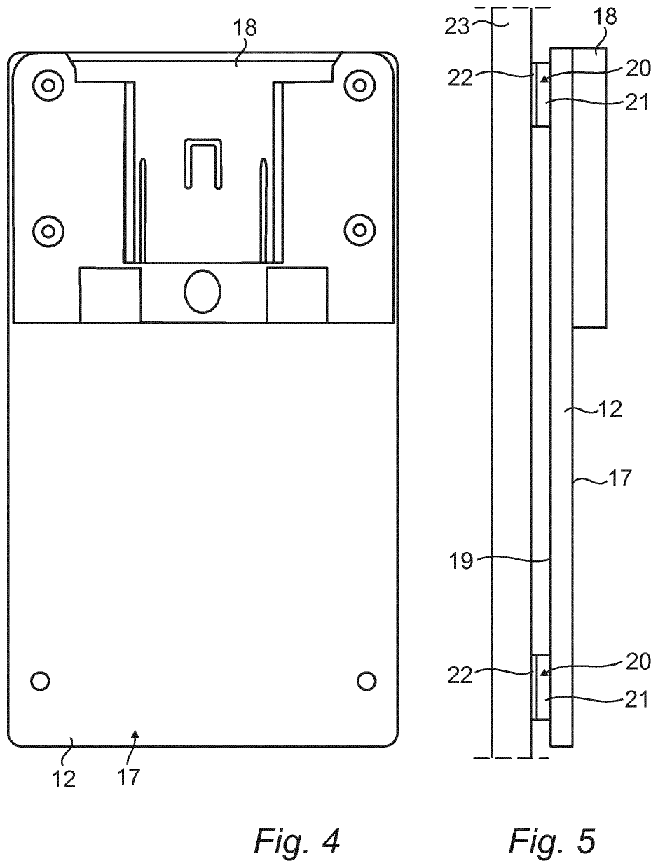

The support plate 12 is arranged for supporting the earplug dispenser 11. The support plate 12 has a front side 17 for carrying the earplug dispenser 11. In the illustrated embodiment, the earplug dispenser 11 is attached to the front side 17 of the support plate 12 through a mounting bracket 18, which is illustrated in FIG. 2 and FIGS. 4 and 5. The support plate 12 comprises a back side 19. For example, the front side 17 and the back side 19 are arranged substantially in parallel. Alternatively, the mounting bracket 18 and the support plate 12 are arranged as an integral unit, for example as a single moulded unit, wherein the mounting bracket is formed in the support plate. The support plate 12, or at least the back side 19 thereof, extends in a plane substantially along the longitudinal axis A. For example, the support plate 12 is substantially rectangular or square, optionally with rounded corners. Alternatively, the support plate 12 is oval or circular.

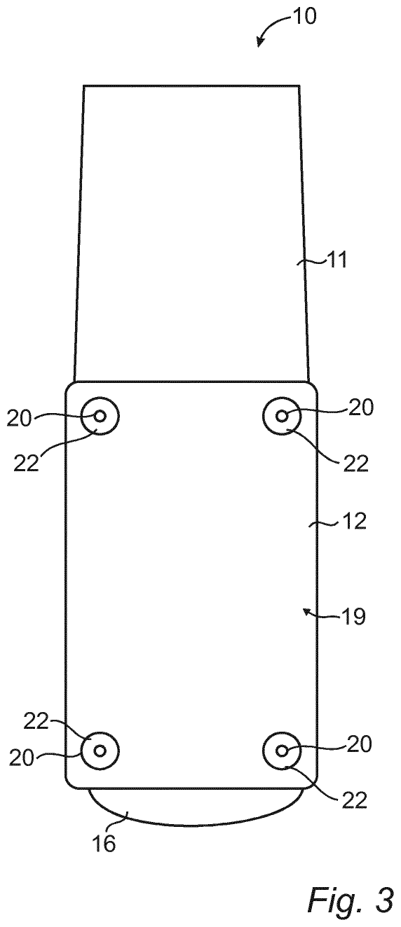

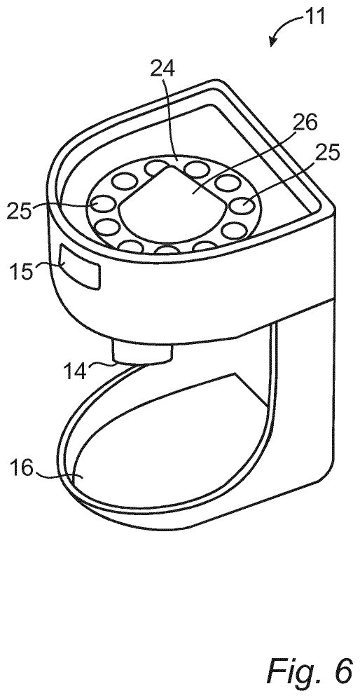

The back side 19 of the support plate 12 is provided with one or more fastening devices 20 comprising a permanent magnet 21 and a friction-increasing material 22 for fastening the support plate 12 to the surface of a vertical element 23 by magnetism in combination with friction, so that the earplug dispensing system 10 is attached to the vertical element 23 by means of the fastening devices 20. The fastening devices 20 are illustrated with reference to FIGS. 2 and 3 and fastening to the vertical element 23 is illustrated in FIG. 5. For example, the earplug dispensing system 10 is attached to the vertical element 23 by means of the fastening devices 20 alone. In the illustrated embodiment, four fastening devices 20 are attached to the support plate 12, wherein the fastening devices 20 are distributed over the surface of the back side. For example, fastening devices 20 are arranged near corners of the support plate 12. Alternatively, any suitable size and number of fastening devices 20 are arranged on the back side 19 of the support plate 12 to attach the earplug dispensing system to the vertical surface by means of magnetic forces and friction. For example, the fastening devices 20 are attached to the support plate 12 by means of screws arranged through holes in the fastening devices 20. Alternatively, the fastening devices 20 are attached to the support plate 12 by means of an adhesive or other suitable fastening means. Alternatively, the support plate 12 comprises a magnetic material, such as iron, wherein the fastening devices 20 are attached to the support plate 12 by means of magnetism through the permanent magnet of the fastening devices 20. For example, the permanent magnets 21 are arranged with a front side and a back side, wherein the front and back sides are substantially plane and level and are arranged substantially in parallel. For example, the permanent magnets 21 are plate shaped. In the illustrated embodiment, the permanent magnets 21 are circular. Alternatively, the permanent magnets 21 are of any suitable shape, such as rectangular, oval, square, etc. The permanent magnets 21 comprises a ferromagnetic material, such as iron, cobalt, nickel, neodymium or other metals, alloys or minerals that can be magnetized to produce a magnetic field for attachment to the vertical element 23 by magnetic forces. At least the back side of the permanent magnet 21 is provided with the friction-increasing material 22, wherein the friction-increasing material 22 is arranged for engaging the surface of the vertical element 23 and preventing the earplug dispensing system 10 from displacement in a plane of the vertical element 23. The friction-increasing material 22 is arranged as a layer or a sheet on the back side of the permanent magnet 21, so that the friction-increasing material 22 is arranged between the permanent magnet 21 and the vertical element 23 when the earplug dispensing system 10 is attached to the vertical element 23. Alternatively, the permanent magnet 21 is covered by the friction-increasing material 22. The friction-increasing material 22 is arranged with higher friction than the permanent magnet 21 to prevent the fastening devices 20 from sliding on the surface of the vertical element 23. For example, the friction-increasing material has higher dry friction, such as a higher static friction coefficient, against the surface of the vertical element 23 than the permanent magnet 21. For example, the friction-increasing material 22 is made of or comprises a high friction polymer, such as an elastomer. For example, the friction-increasing material 22 is or comprises rubber or similar material having higher static friction coefficient against the surface of the vertical element 23 than the permanent magnet 21. The permanent magnet 21 and the friction-increasing material 22 are arranged to attach the fully loaded earplug dispensing system 11 to the vertical element 23 and prevent the fully loaded earplug dispensing system 11 from sliding along the surface of the vertical element 23 due to gravity.

With reference to FIG. 6 the earplug dispenser 11 is illustrated according to one embodiment. Hence, in FIG. 6 the container 13, support plate 12 and mounting bracket 18 have been removed. In the embodiment of FIG. 6 the earplug dispenser 11 comprises the dispensing aperture 14, the control device 15, the optional tray 16 and the dispensing mechanism for dispensing earplugs when the control device 15 is operated. In the embodiment of FIG. 6 the dispensing mechanism comprises a rotatable element 24 having a plurality of through holes 25 for receiving earplugs, such as a single earplug or a pair of earplugs for each hole 25, wherein one of said holes 25 is aligned with the dispensing aperture 14 for dispensing the earplug or earplugs inside the hole 25 when the hole 25 is aligned with the dispensing aperture 14. The rotatable element 24 is arranged below the container 13 or inside the lower part of the container 13 when the container 13 is mounted on top of the dispensing mechanism, so that earplugs are fed into the holes 25 by means of gravity. Hence, the container 13 is open towards the dispensing mechanism. The rotatable element 24 is arranged in contact with earplugs. For example, the rotatable element 24 is arranged in contact with the earplugs in the lowermost part of the container 13. The rotatable element 24 is controlled by the control device 15 for rotating the rotatable element 24, wherein the earplug or earplugs in the hole 25 coming into alignment with the dispensing aperture 14 exits the earplug dispenser 11 by means of gravity while earplugs in the container 13 are received in the empty holes 25. The holes 25 are distributed around the periphery of the rotating element. For example, the rotating element 24 is formed with a tapered or conical centre portion 26 for guiding earplugs towards the holes 25. Alternatively, the earplug dispenser 11 comprises a movable closing element, such as a hinged hatch or similar, operably arranged between a closed position for closing the dispensing aperture 14 and an open position for dispensing earplugs from the container 13 through the dispensing aperture 14. The dispensing aperture 14 extends, for example in a plane perpendicular to the longitudinal axis A. According to one embodiment, the dispensing mechanism is mechanical. Alternatively, the dispensing mechanism is electrical, wherein the dispensing mechanism is connected to an electric motor for operating it, such as for rotating the rotatable element 24 or opening and closing the movable closing element.

1. An earplug dispensing system, comprising an earplug dispenser and a support plate for carrying the earplug dispenser, wherein the earplug dispenser comprises a container for a plurality of earplugs, a dispensing mechanism having a dispensing aperture and a movable element operably arranged for selectively dispensing earplugs from the container through the aperture, and a control device for operating the dispensing mechanism, wherein the support plate on a front side is provided with the earplug dispenser and on a back side is provided with one or more fastening devices for fastening the support plate to a vertical surface, characterized in that the one or more fastening devices comprise a permanent magnet for fastening the support plate with the earplug dispenser to the vertical surface, wherein the magnet has a back side arranged substantially in parallel to the back side of the support plate, and that at least the back side of the permanent magnet is provided with a friction-increasing material for engaging the vertical surface and preventing the earplug dispensing system from displacement in a plane of the vertical surface.

2. The earplug dispensing system according to claim 1, wherein the back side of the support plate is provided with a plurality of fastening devices distributed over the surface of the back side of the support plate.

3. The earplug dispensing system according to claim 1, wherein the permanent magnet is arranged as a plate having a front side arranged in parallel to the back side of the permanent magnet and engaging the back side of the support plate.

4. The earplug dispensing system according to claim 1, comprising a mounting bracket attached to the support plate and comprising a locking element for locking the dispenser to the mounting bracket.

5. The earplug dispensing system according to claim 1, wherein the friction-increasing material is a sheet of elastomer material.

6. The earplug dispensing system according claim 5, wherein the elastomer material is rubber.

7. The earplug dispensing system according to claim 1, wherein the dispenser is arranged substantially vertically, so that the earplugs are dispensed downwards by means of gravity.

8. The earplug dispensing system according to claim 7, wherein the container (13) is arranged substantially vertically.

9. The earplug dispensing system according to claim 2, wherein the permanent magnet is arranged as a plate having a front side arranged in parallel to the back side of the permanent magnet and engaging the back side of the support plate.

10. The earplug dispensing system according to claim 2, comprising a mounting bracket attached to the support plate and comprising a locking element for locking the dispenser to the mounting bracket.

11. The earplug dispensing system according to claim 3, comprising a mounting bracket attached to the support plate and comprising a locking element for locking the dispenser to the mounting bracket.

12. The earplug dispensing system according to claim 2, wherein the friction-increasing material is a sheet of elastomer material.

13. The earplug dispensing system according to claim 3, wherein the friction-increasing material is a sheet of elastomer material.

14. The earplug dispensing system according to claim 4, wherein the friction-increasing material is a sheet of elastomer material.

15. The earplug dispensing system according to claim 2, wherein the dispenser is arranged substantially vertically, so that the earplugs are dispensed downwards by means of gravity.

16. The earplug dispensing system according to claim 3, wherein the dispenser is arranged substantially vertically, so that the earplugs are dispensed downwards by means of gravity.

17. The earplug dispensing system according to claim 4, wherein the dispenser is arranged substantially vertically, so that the earplugs are dispensed downwards by means of gravity.

18. The earplug dispensing system according to claim 5, wherein the dispenser is arranged substantially vertically, so that the earplugs are dispensed downwards by means of gravity.

19. The earplug dispensing system according to claim 6, wherein the dispenser is arranged substantially vertically, so that the earplugs are dispensed downwards by means of gravity.
